(19) United States
(12) Patent Application Publication
(10) Pub. No.: US 2001/0036469 A1
(43) Pub. Date: Nov. 1, 2001

(54) Opiate antagonist implant and process for preparation therefor

(76) Inventor: Lance L. Gooberman, Haddonfield, NJ (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 09/866,978

(22) Filed: May 29, 2001

Related U.S. Application Data
(63) Continuation of application No. 09/644,091, filed on Aug. 23, 2000, now abandoned.
Application No. 09/644,091 is a division of application No. 08/829,003, filed on Mar. 31, 1997, now abandoned.
(60) Provisional application No. 60/028,605, filed on Jan. 13, 1997.

(51) Int. Cl. A61K 9/00; A61K 31/485

(52) U.S. Cl. 424/426; 514/282

(57) ABSTRACT

The present invention provides an opiate antagonist implant which is an admixture of an opiate antagonist, in either acid or base form, and a pharmaceutically acceptable carrier. The admixture is uniformly compressed into a subcutaneously implantable pellet which is effective to release levels of the opiate antagonist over desired amounts of time when subcutaneously implanted in a patient to effectively inhibit the effects of a number of addictive drugs.

### RELATED APPLICATION

This application is a divisional application of U.S. application Ser. No. 08/829,003, which claims the benefit of U.S. Provisional Application Ser. No. 60/028,605, filed on Oct. 23, 1996, entitled NALTREXONE IMPLANT COMPOSITION AND PROCESS FOR PREPARATION THEREFORE.

### FIELD OF THE INVENTION

The present invention relates to an opiate antagonist implant and, more particularly, to such an implant which, when subcutaneously implanted in a patient, will deliver an effective and desired level of an opiate antagonist in the patient's bloodstream for an extended period of time, preferably in excess of thirty days or more. The invention also relates to a process for preparing such an opiate antagonist implant.

### BACKGROUND OF THE INVENTION

Heroin addiction is a growing health care problem in the United States. The United States Department of Health and Human Services' Substance Abuse Branch issued a report in December of 1994 stating that the number of emergency department visits directly related to heroin use rose from 48,000 in 1992 to 63,000 in 1993, a 31% increase. The rate of heroin-related episodes per 100,000 people rose 81%, from 15 to 28 per 100,000, between 1990 and 1993. Upon breaking down the heroin-using population into ethnic groups and age groups, it has been demonstrated that all subsets have increased rates of use for this time period.

Human opiate detoxification has been in use for some time. More than 31,000 individuals of the Empire Blue Cross and Blue Shield subscriber base in New York were hospitalized at least once for opiate dependency between 1982 and 1992. The majority of these individuals were working adults, and their principal reason for hospitalization was addiction treatment. Drug detoxification accounted for 96% of the admissions, and the length of stay ranged between five and ten days.

In cases where individuals have been recently “detoxed” there is a high incident of relapse and readdiction. While these former addicts are often strongly motivated to seek treatment and relapses often produce guilt and depression, they are still unable to resist giving in to the intense craving for heroin or pressure from drug dealers.

In recognition of the foregoing, opiate antagonists have been administered to such detoxified addicts. Opiate antagonists are defined as chemical compounds which block the effects of opiate drugs by blocking the opiate receptors in a patient. By blocking the effects of agonist opiates, opiate antagonists also prevent the development of physical dependence and tolerance to opiate drugs, such as heroin.

It should be noted that while opiate antagonists do not produce symptoms when they are used in the treatment of heroin dependence, they will precipitate an abstinence syndrome in individuals who are physically dependent on an opiate drug. By virtue of their affinity for the opiate receptors, they will compete with and oftentimes displace opiate agonists from the receptor sites. Accordingly, a heroin addict must be detoxified before he can be treated with an opiate antagonist. Once completely free of opiate drugs, however, no symptoms will be produced by the administration of the opiate antagonist.

One preferred antagonist used in the treatment of former heroin addicts is naltrexone. Naltrexone, like all opiate antagonists, provides no euphoric effects and there are no observable pharmacological consequences when a patient stops taking the drug. For naltrexone treatment to be effective, sufficient levels of the drug must be maintained in the patient for a substantial period of time. This typically requires the patient to self-administer dosages of the drug several times a week.

A major problem with the use of opiate antagonists, such as naltrexone, in the treatment of opiate addiction has been patient compliance. This is frequently due to the patient's strong desire to experience the euphoric feeling which would otherwise be prevented by the presence of the opiate antagonist in his or her bloodstream.

One solution for improving patient compliance is the controlled release of naltrexone over a desirably long period of time. One conventional timed release method utilizes the implantation of beads, which contain a physical blend of naltrexone and biodegradable copolymers of lactic acid and glycolic acid. A drawback with this system is that a high level of naltrexone is released rather quickly into the patient's bloodstream resulting in tissue irritation. Further, the process involved in manufacturing the beads is relatively complex and thus excessively costly.

### SUMMARY OF THE INVENTION

One aspect of the present invention comprises the placement of a subcutaneously implantable opiate antagonist in a patient in need thereof to effectively block the effects of heroin and/or other opiates present in the patient for an extended period of time to eliminate the craving response of addicts. This implant is also useful in blocking the positive reinforcement from a number of other addictive substances including cocaine, alcohol, and nicotine. The opiate antagonist implant is subcutaneously implantable in a human body and is adapted to maintain sufficiently high levels of an opiate antagonist in a patient for an extended period of time, preferably at least about thirty days and more, in order to prevent a former addict from relapsing.

The implant comprises an admixture of an opiate antagonist and a pharmaceutically acceptable carrier. The admixture is compressed, preferably uniformly, into a subcutaneously implantable pellet, wherein such compressed admixture is effective to deliver therapeutically effective levels of an opiate antagonist when subcutaneously implanted over an extended period of time in a patient. The implant in accordance with this invention been shown to provide levels of effective opiate antagonist delivery for up to thirty days and longer.

### DETAILED DESCRIPTION OF THE PREFERRED EMBODIMENTS

The present invention is directed toward an opiate antagonist implant in the form of a pellet for delivering an effective amount of an opiate antagonist over a prolonged or extended period of time, preferably in excess of thirty days or more. The implant is adapted to be implanted subcutaneously. The opiate antagonist implant comprises an admixture of an opiate antagonist and a pharmaceutically acceptable carrier, and is prepared by compressing the admixture into a subcutaneously implantable pellet as more fully described hereinbelow. The implanted pellet is adapted to release effective amounts of the opiate antagonist when subcutaneously implanted in a patient over an extended period of time, preferably thirty days or more. While implanted in a patient, the pellet dissolves, releasing opiate antagonist to thereby block the effects of opiates on the human nervous system. The implant is effective in inhibiting the effects of endogenous, exogenous, synthetic and natural opiates, and is effective in inhibiting the effects of a number of other addictive substances including cocaine, alcohol and nicotine.

A variety of opiate antagonists can be utilized in accordance with this invention. Representative examples of such opiate antagonists include, but are not limited to, naltrexone, naloxone, cyclazocine, diprenorphine, metazocine, levalorphan, metazocine, nalorphine, nalmefene, and salts thereof. The preferred opiate antagonist is naltrexone, which has received FDA approval for use in humans and has been shown to be free of severe side-effects. Naltrexone is neither addicting nor habit forming.

Any pharmaceutically acceptable carrier may be used in accordance with this invention, including without limitation, magnesium stearate, stearic acid, starch, and cellulose. The preferred carrier is magnesium stearate which is often used as both a lubricant and a binder in tablets.

A preferred method for manufacturing an opiate antagonist implant, which includes naltrexone as the opiate antagonist and magnesium stearate as the carrier is described in Example I below. It is to be understood however, that this Example is for illustrative purposes only and is not intended to limit the scope of this invention or the claims in any way.

### EXAMPLE I

### PREPARATION

A quantity of naltrexone hydrochloric acid is mixed with purified water in the ratio of 1 gram of naltrexone HCL for every 3 ml of water in order to obtain a slurry. A 10% sodium hydroxide solution is then added to and mixed with the slurry in a ratio of 1.06 ml for every gram of naltrexone HCL in the slurry.

Thereafter, the liquid from the slurry is filtered off using conventional filtering techniques to obtain a solid residue of naltrexone. The residue is then placed between layers of filter paper. The layers of filter paper are pressed in order to absorb excess moisture from the residue. The thus obtained naltrexone product is then dried by heating the same for approximately 24 hours at a constant temperature of approximately 50° C. (120° F.) to obtain a partially dried naltrexone cake. The naltrexone cake is then broken into smaller pieces and subjected to further drying by heating the same for an additional 24 to 48 hours at a constant temperature of approximately 50° C. (120° F.). In an alternative embodiment, naltrexone base can be employed initially instead of converting naltrexone HCL to the naltrexone base.

The dried naltrexone is then triturated in order to reduce the particle size. The carrier, magnesium stearate, is then mixed with the naltrexone in a ratio of 1 mg of magnesium stearate for every 15 mg of naltrexone. The resultant mixture is then dried by heating the same for 24 hours at a constant temperature of 50° C. (120° F.) and then triturated to obtain a homogenous mixture.

Once dried, the naltrexone and magnesium stearate mixture is uniformly compressed into pellets of equal density. This is preferably accomplished by using a hand operated pellet press such as the Parr Pellet Press, Model # Parr. To obtain a pellet of one preferred size, approximately 1.079 grams of the naltrexone/magnesium stearate 15:1 mix is weighed out and then pressed using a ½″ die and punch. The pellet press used preferably applies approximately 1000 psi of pressure on the punch which causes a firm pellet having a homogenous density to be formed. The resulting implantable pellet is preferably cylindrical in shape, has a diameter of approximately 13 mm, has a length of 8 mm, weighs approximately 1.04 grams, has a naltrexone content of approximately 1.006 grams and has a hardness in the range of about 12.5 to about 14.0 kiloponds. In accordance with a preferred embodiment of the invention, a pellet having such properties is expected to deliver therapeutically effective amounts of naltrexone in a patient in which the pellet has been subcutaneously implanted for approximately one month or longer to effectively inhibit the effects of a number of addictive drugs including heroin, cocaine, alcohol, and nicotine.

Once the pellet is removed from the press, it is preferably placed in a 7.5 cm (3″)×10 cm (4″) Mylar pouch which is then heat sealed. The pellet is then preferably gamma irradiated at a minimum of 3 kilogray and a maximum of 6 kilogray of radiation.

### EXAMPLE II

### CLINICAL USE

In use, the pellitized opiate antagonist is typically subcutaneously implanted in a former addict once he or she has successfully completed a detoxification program. Once implanted, opiate antagonist will be released into the patient's bloodstream for an extended period of time. Such time will depend on the size of the pellet inserted in addition to the type and the percentage of the opiate antagonist contained therein. For example, a pellet comprising approximately 95% naltrexone, which is approximately 8 mm long and has a diameter of approximately 13 mm, will supply therapeutic amounts of the opiate antagonist for up to one month or longer. The delivery of sufficient levels of the opiate antagonist in the patient eliminates the mood-altering effects of any opiate that the patient takes, and will help to maintain sobriety while the patient seeks counseling. It has also been discovered that the implant is also useful in eliminating the mood-altering effects of cocaine, alcohol, and nicotine. To increase the period of time in which naltrexone will be effectively delivered into a patient's bloodstream and to effectively block the positive reinforcement from the particular drug, larger pellets may be manufactured and implanted.

The following Table I summarizes the effect of therapeutically effective blocking normally lethal does of 5 cc of Fetanyl IV on eight patients subcutaneously implanted with a compressed a pellitized opiate antagonist composition of the present invention.

Table I
Fentanyl Challenges Post - Implant

| | | | | # of | | | | | |
|---|---|---|---|---|---|---|---|---|---|
| | | | Chall- | Days | Pupillary | | | | |
| | | | enge | Post- | Size (mm) | | Rsp. Rate | | |
| Patient | Age | Sex | Date | Implant | pre | post | pre | post | Response |
| 1 | 30 | m | 12-16-96 | 30 | 2-3 | 2-3 | 18 | 18 | none |
| 2 | 36 | f | 12-18-96 | 31 | 3-4 | 3-4 | 16 | 18 | slight |
| | | | | | | | | | dizzi- |
| | | | | | | | | | ness, |
| | | | | | | | | | light- |
| | | | | | | | | | headed 1 |
| | | | | | | | | | minute |
| | | | | | | | | | post- |
| | | | | | | | | | inject- |
| | | | | | | | | | ion |
| 3 | 33 | f | 12-30-96 | 38 | 2-3 | 2-3 | 16 | 16 | no |
| | | | | | | | | | change, |
| | | | | | | | | | no |
| | | | | | | | | | effect, + |
| | | | | | | | | | pus |
| 4 | 28 | f | 01-02-97 | 28 | 2-3 | 2-3 | 20 | 20 | c/o |
| | | | | | | | | | light- |
| | | | | | | | | | headed, |
| | | | | | | | | | other- |
| | | | | | | | | | wise |
| | | | | | | | | | negative |
| 5 | 28 | m | 01-02-97 | 28 | 2-3 | 2-3 | 20 | 20 | + |
| | | | | | | | | | nausea, |
| | | | | | | | | | possible |
| | | | | | | | | | vagal |
| | | | | | | | | | re- |
| | | | | | | | | | ponse, |
| | | | | | | | | | other- |
| | | | | | | | | | wise |
| | | | | | | | | | negative |
| 6 | 36 | m | 01-03-97 | 37 | 2-3 | 2-3 | 20 | 20 | |
| 7 | 39 | m | 01-10-97 | 30 | 2-3 | 2-3 | 16 | 20 | none |
| 8 | 17 | m | 01-13-97 | 30 | 3-5 | 3-4 | 16 | 16 | slight |
| | | | | | | | | | dizzi- |
| | | | | | | | | | ness, |
| | | | | | | | | | slight |
| | | | | | | | | | pupil- |
| | | | | | | | | | lary |
| | | | | | | | | | change |
| 9 | 30 | m | 01-20-97 | 38 | 2-3 | 2-3 | 20 | 20 | none |
| 10 | | f | 01-23-97 | 38 | 2-3 | 2-3 | 20 | 20 | none |
| 11 | | | 01-28-97 | 62 | 2-3 | 2-3 | 20 | 20 | none |
| 12 | | | 02-05-97 | 41 | 3 | 3 | 18 | 18 | none |
| 13 | | | 02-10-97 | 75 | 2-3 | 2-3 | 20 | 20 | none |
| 14 | | m | 02-11-97 | 22 | 2-3 | 2-3 | 16 | 16 | none |

The present invention may be embodied in other specific forms without departing from the spirit or essential attributes thereof. Accordingly, reference should be made to the appended claims rather than the foregoing specification as indicating the scope of the invention.

1. An opiate antagonist implant comprising an admixture of an opiate antagonist and a pharmaceutically acceptable carrier, said admixture being compressed into a subcutaneously implantable pellet, wherein said implantable pellet is effective to release said opiate antagonist over time when subcutaneously implanted in a patient.

2. The opiate antagonist implant of claim 1 wherein said implantable is effective to release levels of said opiate antagonist over an extended period of time when subcutaneously implanted in a patient.

3. The opiate antagonist implant of claim 2 where said extended period of time is at least approximately 30 days.

4. The opiate antagonist of claim 1 wherein said opiate antagonist is selected from the group consisting of an endogenous, exogenous, synthetic and natural opiate antagonist.

5. The opiate antagonist implant of claim 1 wherein said opiate antagonist is selected from the group consisting of naltrexone, naloxone, nalmefene, cyclazocine, diprenorphine, metazocine, levalorphan, metazocine, nalorphine, and salts thereof.

6. The opiate antagonist implant of claim 1 wherein said pharmaceutically acceptable carrier is magnesium stearate.

7. The opiate antagonist implant of claim 1 wherein said subcutaneously implantable pellet is cylindrical in shape, is approximately 8 mm long and has a diameter of approximately 13 mm.

8. The opiate antagonist implant of claim 1 wherein said opiate antagonist comprises approximately 95% of the implant by weight.

9. The opiate antagonist implant of claim 1 wherein said subcutaneously implantable pellet has a hardness of in the range of about 12 to about 15 kiloponds.

10. A process of manufacturing an opiate antagonist implant comprising compressing a therapeutically effective amount of an opiate antagonist with a pharmaceutically acceptable carrier.

11. The process of claim 10 wherein the amount of said compression produces an implant which is effective to release said opiate antagonist in therapeutically effective amounts for at least about 30 days or longer.

12. The process of claim 10 wherein said implant has a moisture content which is effective to release said opiate antagonist in therapeutically effective amounts for at least about 30 days or longer.

13. A process of manufacturing a naltrexone implant comprising the steps of:
(a) providing a quantity of naltrexone;
(b) providing a pharmaceutically acceptable carrier;
(c) admixing said pharmaceutically acceptable carrier and said naltrexone in a ratio of approximately 15 grams of said naltrexone for approximately every one gram of said pharmaceutically acceptable carrier;
(d) triturating the admixture of step (c);
(e) applying uniform pressure to the triturated admixture of step (d) in order to obtain a naltrexone implant in the form of a firm pellet.

14. The method of claim 13 wherein approximately 1000 psi of pressure is applied to the triturated admixture of step (d).

15. The method of claim 13 wherein said triturated admixture of step (d) is dried for approximately 24 hours at a constant temperature of approximately 50° C. (120° F.).

16. A process of manufacturing a naltrexone implant comprising the steps of:
(a) providing a quantity of naltrexone HCL;
(b) providing a quantity of water;
(c) admixing said quantity of water with said quantity of naltrexone HCL in a ratio of approximately 3 ml of water for approximately every one gram of naltrexone HCL until a slurry is obtained;
(d) providing a quantity of 10% sodium hydroxide solution;
(e) admixing said quantity of 10% sodium hydroxide solution to said slurry of step (c) in the ratio of approximately 1 ml of sodium hydroxide for approximately every one gram of naltrexone HCL;
(f) filtering off any liquid from said slurry to obtain a solid residue of naltrexone;
(g) drying said residue of naltrexone for approximately 24 to approximately 72 hours at a constant temperature of 50° C. (120° F.);
(h) providing a pharmaceutically acceptable carrier;
(i) admixing said pharmaceutically acceptable carrier and said naltrexone in a ratio of approximately 15 grams of said naltrexone for approximately every one gram of said pharmaceutically acceptable carrier;
(j) triturating said admixture of step (i), and (k) applying uniform pressure to said triturated admixture of step (j) in order to obtain a naltrexone implant in the form of a firm pellet.

17. The method of claim 16 wherein approximately 1000 psi of pressure is applied to the triturated admixture of step (j)

18. A method for administering predetermined, effective amounts of an opiate antagonist from an implant to a human which comprises subcutaneously implanting the implant of claim 1 in a human so that the human receives controlled amounts of said opiate antagonist for an extended period of time.

19. The method of claim 18 wherein said implant comprises said opiate antagonist in an amount of approximately 95% by weight and said pharmaceutically acceptable carrier in an amount of approximately 5% by weight.

20. The method of claim 18 wherein said opiate antagonist is selected from the group consisting, of naltrexone, naloxone, nalmefene, cyclazocine, diprenorphine, etazocine, levalorphan, metazocine, nalorphine, and salts thereof.

21. The method of claim 18 wherein said pharmaceutically acceptable carrier is magnesium stearate.

22. The method of claim 18 wherein said extended period of time is at least approximately 30 days.

23. A method of treating an opiate addicted patient for opiate addiction comprising administering subcutaneously an opiate antagonist implant comprising an opiate antagonist implant comprising an admixture of an opiate antagonist and a pharmaceutically acceptable carrier, said admixture being compressed into a subcutaneously implantable pellet, wherein said implantable pellet is effective to release levels of said opiate antagonist over time when subcutaneously implanted in a patient.

24. A method of using an opiate antagonist implant comprising an opiate antagonist implant comprising an admixture of an opiate antagonist and a pharmaceutically acceptable carrier, said admixture being compressed into a subcutaneously implantable pellet, wherein said implantable pellet is effective to release levels of said opiate antagonist over time when subcutaneously implanted in a patient by subcutaneously implanting said implant in a patient in need thereof.
